(19) United States
(12) Patent Application Publication
(10) Pub. No.: US 2009/0062246 A1
(43) Pub. Date: Mar. 5, 2009

(54) THERAPEUTIC TREATMENT-014

(75) Inventor: Pia Wulfing, Munster (DE)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/192,427

(22) Filed: Aug. 15, 2008

Related U.S. Application Data
(60) Provisional application No. 61/036,126, filed on Mar. 13, 2008.
(60) Provisional application No. 60/956,628, filed on Aug. 17, 2007.

(51) Int. Cl. A61K 31/565; A61K 31/497

(52) U.S. Cl. 514/171; 514/255.05

(57) ABSTRACT

A combination, comprising N-(3-methoxy-5-methylpyrazin-2-yl)-2-(4-[1,3,4-oxadiazol-2-yl]phenyl)pyridine-3-sulphonamide, or a pharmaceutically acceptable salt thereof, and an aromatase inhibitor or an estrogen receptor down-regulator is described. The combination is expected to be useful in the treatment of cancer.

### RELATED APPLICATIONS

This application claims the benefit under 35 U.S.C. 119(e) of U.S. Provisional Application No. 60/956,628, filed Aug. 17, 2007 and U.S. Provisional Application No. 61/036,126, filed Mar. 13, 2008. The entire teaching of U.S. 60/956,628 and U.S. 61/036,126 are incorporated herein by reference.

### FIELD OF THE INVENTION

The present invention relates to a combination comprising N-(3-methoxy-5-methylpyrazin-2-yl)-2-(4-[1,3,4-oxadiazol-2-yl]phenyl)pyridine-3-sulphonamide, or a pharmaceutically acceptable salt thereof, hereafter “Compound (I)”, and an aromatase inhibitor. The present invention further relates to a combination comprising Compound (I) and an estrogen receptor down-regulator.

These combinations are useful for the treatment or prophylaxis of cancer. The invention also relates to a pharmaceutical composition comprising such combinations and to the use thereof in the manufacture of a medicament for use in the treatment or prophylaxis of cancer, in particular prostate cancer.

### BACKGROUND OF THE INVENTION

Cancer affects an estimated 10 million people worldwide. This figure includes incidence, prevalence and mortality. More than 4.4 million cancer cases are reported from Asia, including 2.5 million cases from Eastern Asia, which has the highest rate of incidence in the world. By comparison, Europe has 2.8 million cases, North America 1.4 million cases, and Africa 627,000 cases.

In the UK and US, for example, more than one in three people will develop cancer at some point in their life. Cancer mortality in the U.S. is estimated to account for about 600,000 a year, about one in every four deaths, second only to heart disease in percent of all deaths, and second to accidents as a cause of death of children 1-14 years of age. The estimated cancer incidence in the U.S. is now about 1,380,000 new cases annually, exclusive of about 900,000 cases of non-melanotic (basal and squamous cell) skin cancer.

Cancer is also a major cause of morbidity in the UK with nearly 260,000 new cases (excluding non-melanoma skin cancer) registered in 1997. Cancer is a disease that affects mainly older people, with 65% of cases occurring in those over 65. Since the average life expectancy in the UK has almost doubled since the mid nineteenth century, the population at risk of cancer has grown. Death rates from other causes of death, such as heart disease, have fallen in recent years while deaths from cancer have remained relatively stable. The result is that 1 in 3 people will be diagnosed with cancer during their lifetime and 1 in 4 people will die from cancer. In people under the age of 75, deaths from cancer outnumber deaths from diseases of the circulatory system, including ischaemic heart disease and stroke. In 2000, there were 151,200 deaths from cancer. Over one fifth (22 percent) of these were from lung cancer, and a quarter (26 percent) from cancers of the large bowel, breast and prostate.

Worldwide, the incidence and mortality rates of certain types of cancer (of stomach, breast, prostate, skin, and so on) have wide geographical differences which are attributed to racial, cultural, and especially environmental influences. There are over 200 different types of cancer but the four major types, lung, breast, prostate and colorectal, account for over half of all cases diagnosed in the UK and US. Prostate cancer is the fourth most common malignancy among men worldwide, with an estimated 400,000 new cases diagnosed annually, accounting for 3.9 percent of all new cancer cases.

Current options for treating cancers include surgical resection, external beam radiation therapy and/or systemic chemotherapy. These are partially successful in some forms of cancer, but are not successful in others. There is a clear need for new therapeutic treatments.

Recently, endothelin A receptor antagonists have been identified as potentially of value in the treatment of cancer (Cancer Research, 56, 663-668, Feb. 15^{th}, 1996 and Nature Medicine, Volume 1, Number 9, September 1999, 944-949).

The endothelins are a family of endogenous 21 amino acid peptides comprising three isoforms, endothelin-1, endothelin-2 and endothelin-3. The endothelins are formed by cleavage of the Trp²¹-Val²² bond of their corresponding proendothelins by an endothelin converting enzyme. The endothelins are among the most potent vasoconstrictors known. They exhibit a wide range of other activities including stimulation of cell proliferation and mitogenesis, inhibition of apoptosis, extravasation and chemotaxis, and also interact with a number of other vasoactive agents.

The endothelins are released from a range of tissue and cell sources including vascular endothelium, vascular smooth muscle, kidney, liver, uterus, airways, intestine and leukocytes. Release can be stimulated by hypoxia, shear stress, physical injury and a wide range of hormones and cytokines. Elevated endothelin levels have been found in a number of disease states in man including cancers.

Endothelins exert their effects by binding via two G-protein-coupled receptors—endothelin receptor A and B (ET_{A} and ET_{B}). ET_{A} and ET_{B} appear to influence tumour progression by several mechanisms, including cell proliferation, inhibition of apoptosis, angiogenesis, matrix remodelling, and bone deposition in skeletal metastases through activation of osteoblasts (Nelson et al, Nat Rev Cancer 2003; 3: 110-116; Bagnato et al, Trends Endocrinol. Metab. 2002; 14: 44-50; and Rosano et al, Cancer Res 2001; 61: 8340-8346).

Activation of ET_{A} by endothelin-1 (ET-1) promotes tumour growth and progression by inhibiting apoptosis, synergizing with other growth factors to cause cell proliferation, and by stimulating the production of the key angiogenic factor VEGF in response to hypoxia (Bagnato et al, Trends Endocrinol. Metab. 2002; 14: 44-50). ET_{A} activation also induces matrix-degrading enzymes, such as matrix metalloproteinases and urokinase plasminogen activator, which have important roles in tissue remodelling and tumour metastasis (Rosano et al, Cancer Res 2001; 61: 8340-8346). In neuronal cells, ET-1/ET_{A} binding is involved in nociceptive effects associated with cancer bone metastasis and remodelling, and thus may be associated with bone pain in patients with bone metastasis.

In contrast, activation of ET_{B} by ET-1 promotes vasodilation and induces apoptosis in human cancer cells (Okazawa et al, J. Biol. Chem. 1998; 273: 12584-12592). In addition, following activation of ET_{B}, the endothelin-ET_{B} complex is internalized, which in turn decreases the concentration of endothelin in the blood.

Therefore a compound that specifically inhibits ET_{A} receptor binding whilst having no effect on endothelin-1 binding to ET_{B} would be expected to inhibit the negative tumour pathology associated with ET_{A} receptor signalling whilst not affect the beneficial ET_{B} receptor signalling. Compound (I) is such a specific ET_{A} receptor antagonist.

Compound (I) is exemplified and described in WO96/40681 as Example 36. WO96/40681 claims the endothelin receptor antagonists described therein for the treatment of cardiovascular diseases. The use of Compound (I) in the treatment of cancers and pain is described in WO04/018044.

Compound (I) has the following structure:

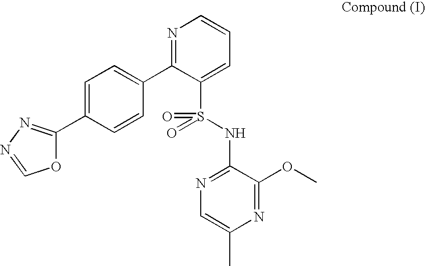

In WO04/018044 an endothelin human receptor binding assay is described. The pIC₅₀ (negative log of the concentration of compound required to displace 50% of the ligand) for Compound (I) at the ET_{A} receptor was 8.27 [8.23-8.32] (n=4). In contrast Compound (I) had no measurable affinity for the ET_{B} receptor. Compound (I) is thus an excellent specific ET_{A} receptor antagonist and is expected to be beneficial in the treatment of cancer. The compound is currently undergoing phase III trials to investigate its effect in the treatment on hormone resistant prostate cancer.

An aromatase inhibitor is a drug that inhibits the enzyme aromatase and by that means lowers the level of the estrogen estradiol. Aromatase is an enzyme of the cytochrome P-450 superfamily and the product of the CYP19 gene, and is highly expressed in the placenta and in the granulosa cells of ovarian follicles, where its expression depends on cyclical gonadotropin stimulation. Aromatase inhibitors represent a class of antiestrogens.

Aromatase catalyzes the conversion of testosterone (an androgen) to estradiol (an estrogen) in many tissues including the adrenal glands, ovaries, placenta, testicles, adipose tissue and brain. Estrogen is produced directly by the ovaries and is also made by the body using aromatase. Aromatase inhibitors interfere with the body's use of aromatase.

The growth of many breast cancers is promoted by estrogens. Most estrogen after menopause comes from the action of aromatase. Aromatase inhibitors may therefore be used to treat estrogen-dependent tumours after the menopause. Aromatase inhibitors are used mostly in women who have reached menopause, when the ovaries are no longer producing estrogen.

The only approved estrogen-receptor down-regulator (ERD) is Faslodex (chemical name: fulvestrant). Faslodex is an option for post-menopausal women with advanced (metastatic) breast cancer that is hormone-receptor-positive and has stopped responding to other anti-estrogen therapy.

Faslodex offers a benefit to post-menopausal women with metastatic (advanced) hormone-receptor-positive breast cancer whose cancer has progressed on either tamoxifen or aromatase inhibitors or who cannot take other hormonal medications (possibly because of other medical conditions).

Faslodex is a competitive inhibitor of estrogen action by binding to the estrogen receptor (ER) and preventing access to estrogen. In addition, the binding of Faslodex to the ER results in a reduction in ER protein levels leading to a rapid degredation of the ER in the target tissue resulting in insufficient ER for binding to estrogen. The net effect is that estrogen cannot exert its biological effect due to the ER being “down-regulated” and inactivated.

WO96/40681 discloses Compound (I) as one of the many examples disclosed therein and indicates that the compounds described therein may be administered with a beta-adrenergic blocker (for example atenolol), a calcium channel blocker (for example nifedipine), an angiotensin converting enzyme (ACE) inhibitor (for example lisinopril), a diuretic (for example furosemide or hydrochlorothiazide), an endothelin converting enzyme (ECE) inhibitor (for example phosphoramidon), a neutral endopeptidase (NEP) inhibitor, an HMGCoA reductase inhibitor, a nitric oxide donor, an anti-oxidant, a vasodilator, a dopamine agonist, a neuroprotective agent, a steroid, a beta-agonist, an anti-coagulant, or a thrombolytic agent. WO2004/032922 discloses combinations on Compound (I) and a 5-HT_{1B/1D} receptor agonist. WO2004/035057 discloses a combination of Compound (I) with an epidermal growth factor receptor tyrosine kinase inhibitor. WO2005/023264 discloses combinations of Compound (I) with an LHRH analogue and/or a bisphosphonate. WO2006/056760 discloses combinations of Compound (I) with an anti-mitotic cytotoxic agent such as a taxane. None of these references disclose a combination of Compound (I) with an aromatase inhibitor or an estrogen receptor down-regulator.

The present inventors have unexpectedly found that:

i) the combination use of Compound (I) and aromatase inhibitors; or
ii) the combination use of Compound (I), and estrogen receptor down-regulators; can have a particular benefit in the treatment of cancer.

### SUMMARY OF THE INVENTION

According to the present invention, there is provided a combination, comprising Compound (I), and an aromatase inhibitor.

According to a further aspect of the present invention, there is provided a combination, comprising Compound (I), and an estrogen receptor down-regulator.

According to a further aspect of the invention, there is provided a method of treating cancer, in a warm-blooded animal, such as man, in need of such treatment which comprises administering to said animal an effective amount of Compound (I) in combination with an effective amount of an aromatase inhibitor.

According to a further aspect of the invention, there is provided a method of treating cancer, in a warm-blooded animal, such as man, in need of such treatment which comprises administering to said animal an effective amount of Compound (I) in combination with an effective amount of an estrogen receptor down-regulator.

According to a further aspect of the invention there is provided a pharmaceutical composition which comprises Compound (I) and an aromatase inhibitor in association with a pharmaceutically acceptable diluent or carrier.

According to a further aspect of the invention there is provided a pharmaceutical composition which comprises Compound (I) and an estrogen receptor down-regulator in association with a pharmaceutically acceptable diluent or carrier.

### DESCRIPTION OF THE FIGURES

FIG. 1A shows Migration in MCF-7aro breast cancer cells—effects of Compound (I) and aromatase inhibitor anastrozole, alone, and in combination with each other. The y-axis shows the relative migration, expressed as a percentage of the cell numbers on compound-treated inserts compared to those of the control.

FIG. 1B shows Migration in MCF-7aro breast cancer cells—effects of Compound (I) and aromatase inhibitor letrozole, alone, and in combination with each other. The y-axis shows the relative migration, expressed as a percentage of the cell numbers on compound-treated inserts compared to those of the control.

FIG. 2 shows Migration in MCF-7 breast cancer cells—effects of Compound (I) and estrogen-receptor down-regulator fulvestrant alone, and in combination with each other. The y-axis shows the relative migration, expressed as a percentage of the cell numbers on compound-treated inserts compared to those of the control.

FIG. 3 shows Invasion in MCF-7 breast cancer cells—effects of Compound (I) and estrogen-receptor down-regulator fulvestrant alone, and in combination with each other. The y-axis shows the relative invasiveness expressed as percentage of the cell numbers on compound-treated inserts compared to control inserts.

FIG. 4 shows inhibition of tumour growth in a post menopausal (ovariectomy) nude mouse xenograft model 45 days after being injected with MCF-7-Acl breast cancer cells—effects of Compound (I), estrogen-receptor down-regulator fulvestrant or an aromatase inhibitor anastrozole alone, and combinations of Compound (I) and estrogen-receptor down-regulator fulvestrant or Compound (I) and an aromatase inhibitor anastrozole.

In all the figures “*” and “**” indicate statistically significant results compared with controls. A “*” represents a p value of <0.05 and a “**” a p value of <0.001.

### DETAILED DESCRIPTION OF THE INVENTION

Herein where the term “aromatase inhibitor” is used it is to be understood that this refers to any chemical compound, or a pharmaceutically acceptable salt thereof, which inhibits the enzyme aromatase and by that means lowers the level of the estrogen estradiol.

An “estrogen receptor down-regulator” is a compound, or a pharmaceutically acceptable salt thereof, which binds to the ER leading to a reduction in ER protein levels and degredation of the ER in the target tissue and by those means prevents estogen from exerting its biological actions.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

In one aspect, where a compound or a pharmaceutically acceptable salt thereof, is referred to this refers to the compound only. In another aspect this refers to a pharmaceutically acceptable salt of the compound.

Where cancer is referred to, particularly it refers to oesophageal cancer, myeloma, hepatocellular, pancreatic, cervical cancer, Ewing's tumour, neuroblastoma, Kaposi's sarcoma, ovarian cancer, breast cancer, colorectal cancer, prostate cancer, bladder cancer, melanoma, lung cancer—non small cell lung cancer (NSCLC), and small cell lung cancer (SCLC), gastric cancer, head and neck cancer, brain cancer, renal cancer, lymphoma and leukaemia. More particularly it refers to prostate cancer. In addition, more particularly it refers to SCLC, NSCLC, colorectal cancer, ovarian cancer and/or breast cancer. In addition, more particularly it refers to SCLC. In addition, more particularly it refers to NSCLC. In addition, more particularly it refers to colorectal cancer. In addition, more particularly it refers to ovarian cancer. In addition, more particularly it refers to breast cancer. In addition, more particularly it refers to hormone receptor positive breast cancer, especially to hormone receptor positive breast cancer in post-menopausal women. In addition, more particularly it refers to metastatic hormone receptor positive breast cancer, especially to metastatic hormone receptor positive breast cancer in post-menopausal women. Furthermore, more particularly it refers to bladder cancer, oesophageal cancer, gastric cancer, melanoma, cervical cancer and/or renal cancer. In addition it refers to endometrial, liver, stomach, thyroid, rectal and/or brain cancer. In another aspect of the invention, the cancer is not melanoma. In another embodiment of the invention, particularly the cancer is in a metastatic state, and more particularly the cancer produces metastases to the bone. In a further embodiment of the invention, particularly the cancer is in a metastatic state, and more particularly the cancer produces skin metastases. In a further embodiment of the invention, particularly the cancer is in a metastatic state, and more particularly the cancer produces lymphatic metastases. In a further embodiment of the invention, the cancer is in a non-metastatic state. In another aspect of the invention the cancer is locally advanced (cancer has spread to tissues close to the site of the primary tumour). In one embodiment the cancer is non-metastatic hormone resistant prostate cancer. In another embodiment the cancer is non-metastatic hormone resistant prostate cancer. In a further embodiment the cancer is metastatic hormone resistant prostate cancer. In a still further embodiment the cancer is estrogen and/or progesterone receptor positive breast cancer. In a further embodiment the cancer is estrogen and/or progesterone receptor positive metastatic breast cancer.

Where the treatment of cancer is referred to particularly this is the treatment of cancerous tumours expressing endothelin A. This treatment is in terms of one or more of the extent of the response (for example reduced tumour volume or reduced tumour burden), the response rate, the clinical benefit rate (the sum of complete response, partial response and stable disease), the time to disease progression and the survival rate (for example progression-free survival and the overall survival rate). Such clinical trial endpoints are well known and are described in for example the FDA publication “Guidance for Industry Clinical Trial Endpoints for the Approval of Cancer Drugs and Biologics” May 2007 (www.fda.gov/CbER/gdlns/clintrialend.htm).

The combinations according to the invention are expected to provide an anti-tumour effect, for example one or more of inhibition of tumour growth, tumour growth delay, regression of tumour, shrinkage of tumour, increased time to re-growth of tumour on cessation of treatment or slowing of disease progression.

It is further expected that the combination use of Compound (I) and particular aromatase inhibitors or particular estrogen receptor down-regulators will have a beneficial effect in preventing the onset of cancer in warm-blooded animals, such as man.

Particular compounds, or pharmaceutically acceptable salts thereof possessing aromatase inhibitor activity include anastrozole, exemestane, letrozole, aminoglutethimide, formestane, fadrozole, rogletimide and vorozole.

In one aspect the aromatase inhibitor is selected from anastrozole. In one aspect the aromatase inhibitor is selected from exemestane. In one aspect the aromatase inhibitor is selected from letrozole. In one aspect the aromatase inhibitor is selected from aminoglutethimide. In one aspect the aromatase inhibitor is selected from formestane. In one aspect the aromatase inhibitor is selected from fadrozole. In one aspect the aromatase inhibitor is selected from rogletimide. In one aspect the aromatase inhibitor is selected from vorozole.

A particular estrogen receptor down-regulator for use in the present invention is fulvestrant. A further particular estrogen receptor down-regulator for use in the present invention is AZD4992. A further particular estrogen receptor down-regulator for use in the present invention is CH-4893237. A further particular estrogen receptor down-regulator for use in the present invention is one of the compounds from U.S. Pat. No. 7,018,994B2, the specific examples of which are incorporated herein by reference.

A further particular estrogen receptor down-regulator for use in the present invention is 11β-fluoro-7α-{5-[methyl(7,7,8,8,9,9,9-heptafluorononyl)amino]pentyl}-17α-methylestra-1,3,5(10)-triene-3,17-β-diol N-oxide.

A further particular estrogen receptor down-regulator for use in the present invention is 11β-fluoro-7α-{5-[methyl(8,8,9,9,10,10,10-heptafluorodecyl)amino]pentyl}-17α-methylestra-1,3,5(10)-triene-3,17β-diol N-oxide.

A further particular estrogen receptor down-regulator for use in the present invention is (RS)-11β-fluoro-7α-{5-[methyl(7,7,8,8,9,9,10,10,10-nonafluorodecyl)amino]-pentyl}-17α-methylestra-1,3,5(10)-triene-3,17β-diol N-oxide.

A further particular estrogen receptor down-regulator for use in the present invention is 11β-fluoro-7α-{5-[methyl(8,8,9,9,9-pentafluorononyl)amino]pentyl}-17α-methylestra-1,3,5(10)-triene-3,17β-diol N-oxide.

A further particular estrogen receptor down-regulator for use in the present invention is 11β-fluoro-7α-{5-[methyl(9,9,10,10,10-pentafluorodecyl)amino]pentyl-17α-methylestra-1,3,5(10)-triene-3,17β-diol N-oxide.

A further particular estrogen receptor down-regulator for use in the present invention is 11β-fluoro-7α-{5-[methyl(8,8,9,9,9-pentafluorononyl)amino]pentyl}-17α-methylestra-1,3,5(10)-triene-3,17β-diol.

A further particular estrogen receptor down-regulator for use in the present invention is 11β-fluoro-7α-{5-[methyl(7,7,8,8,9,9,10,10,10-nonafluorodecyl)amino]pentyl}-17α-methylestra-1,3,5(10)-triene-3,17α-diol.

A further particular estrogen receptor down-regulator for use in the present invention is 11β-fluoro-7α-{5-[methyl(7,7,8,8,9,9,9-heptafluorononyl)amino]pentyl}-17α-methylestra-1,3,5(10)-triene-3,17α-diol.

A further particular estrogen receptor down-regulator for use in the present invention is 17α-ethinyl-11β-fluoro-7α-{5-[methyl(7,7,8,8,9,9,10,10,10-nonafluorodecyl)-amino]pentyl}-estra-1,3,5(10)-triene-3,17β-diol.

A further particular estrogen receptor down-regulator for use in the present invention is 17α-ethinyl-11β-fluoro-3-(2-tetrahydropyranoyloxy)-7α-{5-[methyl(7,7,8,8,9,9,10,10,10-nonafluorodecyl)amino[pentyl}-estra-1,3,5(10)-trien-17β-ol.

A further particular estrogen receptor down-regulator for use in the present invention is 11β-fluoro-3-(2-tetrahydrophyranyloxy)-7α-{5-methyl(7,7,8,8,9,9,10,10,10-nonafluorodecyl)amino]pentyl}-17α-methylestra-1,3,5(10)-trien-17β-ol.

A further particular estrogen receptor down-regulator for use in the present invention is 11β-fluoro-7α-{5-[methyl(7,7,8,8,9,9,10,10,10-nonafluorodecyl)amino]pentyl}-17α-trifluoromethylestra-1,3,5 (10)-triene-3,17β-diol.

A further particular estrogen receptor down-regulator for use in the present invention is 11β-fluoro-7α-{5-[methyl(6,6,7,7,8,8,8-heptafluorooctyl)amino[pentyl}-17αmethylestra-1,3,5(5-triene-3,17β-diol.

A further particular estrogen receptor down-regulator for use in the present invention is 11β-fluoro-7α-{5-[methyl(8,8,9,9,10,10,10-heptafluorodecyl)amino[pentyl}-17α-methylestra-1,3,5(10)-triene-3,17β-diol.

A further particular estrogen receptor down-regulator for use in the present invention is 11β-fluoro-7α-{5-[methyl(6,6,7,7,8,8,9,9,10,10,10-undecafluorodecyl)amino]-pentyl}-17α-methylestra-1,3,5(10)-triene-3,17β-diol.

A further particular estrogen receptor down-regulator for use in the present invention is 11β-fluoro-7α-{5-[methyl(5,5,6,6,7,7,8,8,8-nonafluorooctyl)aminopentyl}-17α-methylestra-1,3,5(10)-triene-3,17β-diol.

A further particular estrogen receptor down-regulator for use in the present invention is 11β-fluoro-7α-{5-[methyl(9,9,10,10,11,11,11-heptafluoroundecyl)amino[pentyl}-17α-methylestra-1,3,5(10)-triene-3,17β-diol.

A further particular estrogen receptor down-regulator for use in the present invention is 11β-7α-{5-[methyl(9,9,10,10,10-pentafluorodecyl)amino]pentyl}-17α-methylestra-1,3,5(10)-triene-3,17β-diol.

A further particular estrogen receptor down-regulator for use in the present invention is 11β-fluoro-7α-{5-[methyl(7,7,8,8,9,9-heptaflorononyl)amino]pentyl}-17αmethylestra-1,3,5(10)-triene-3,17β-diol N-oxide.

A further particular estrogen receptor down-regulator for use in the present invention is 11β-fluoro-7α-{5-(methyl {3-[(2,3,4,5,6-pentafluorophenyl)sulfanyl]propyl}-amino}pentyl]estra-1,3,5(10)-triene-3,17β-diol N-oxide.

A further particular estrogen receptor down-regulator for use in the present invention is 11β-fluoro-7α-[5-(methyl{3-[(4,4,5,5,5-pentafluoropentyl)sulfanyl]propyl}amino)-pentyl]estra-1,3,5(10)-triene-3,17β-diol N-oxide.

A further particular estrogen receptor down-regulator for use in the present invention is 11β-fluoro-7α-[5-(methyl{3-[(4,4,5,5,5-pentafluoropentyl)sulfinyl]propyl}amino)-pentyl]estra-1,3,5(10)-triene-3,17β-diol N-oxide.

A further particular estrogen receptor down-regulator for use in the present invention is 11β-fluoro-7α-{5-[methyl(7,7,8,8,9,9,10,10,10-nonafluorodecyl)amino]pentyl}estra-1,3,5 (10)-triene-3,17β-diol N-oxide.

A further particular estrogen receptor down-regulator for use in the present invention is (S)-11β-fluoro-7α-{5-[methyl(7,7,8,8,9,9,10,10,10-nonafluorodecyl)amino]pentyl}-17α-methylestra-1,3,5(10)-triene-3,17β-diol N-oxide.

A further particular estrogen receptor down-regulator for use in the present invention is (R)-11β-fluoro-7α-{5-[methyl(7,7,8,8,9,9,10,10,10-nonafluorodecyl)amino]pentyl}-17α-methylestra-1,3,5(10)-triene-3,17β-diol N-oxide.

A further particular estrogen receptor down-regulator for use in the present invention is 11β-fluoro-7α-{5-[methyl(9,9,10,10,10-pentylfluorodecyl)amino]pentyl}estra-1,3,5(10)-triene-3,17β-diol N-oxide.

A further particular estrogen receptor down-regulator for use in the present invention is 11β-fluoro-7α-[5-(methyl{3-[(4,4,5,5,5-pentafluoropentyl)sulfinyl]propyl}amino)-pentyl]estra-1,3,5(10)-trien-3-ol-17-one N-oxide.

A further particular estrogen receptor down-regulator for use in the present invention is 11β-fluoro-7α-[5-(methyl{3-[(4,4,5,5,5-pentafluoropentyl)sulfanyl]propyl}amino)-pentyl]estra-1,3,5(10)-trien-3-ol-17-one N-oxide.

A further particular estrogen receptor down-regulator for use in the present invention is 11β-fluoro-7α-{5[methyl(7,7,8,8,9,9,10,10,10-nonafluorodecyl)amino]pentyl}estra-1,3,5 (10)-trien-3-ol-17one N-oxide.

Particular combinations of the present invention include:
Compound (I) and anastrozole, or a pharmaceutically acceptable salt thereof;
Compound (I) and exemestane, or a pharmaceutically acceptable salt thereof;
Compound (I) and letrozole, or a pharmaceutically acceptable salt thereof;
Compound (I) and aminoglutethimide, or a pharmaceutically acceptable salt thereof;
Compound (I) and formestane, or a pharmaceutically acceptable salt thereof;
Compound (I) and fadrozole, or a pharmaceutically acceptable salt thereof;
Compound (I) and rogletimide, or a pharmaceutically acceptable salt thereof;
Compound (I) and vorozole, or a pharmaceutically acceptable salt thereof;
Compound (I) and fulvestrant, or a pharmaceutically acceptable salt thereof;
Compound (I) and AZD4992, or a pharmaceutically acceptable salt thereof; and
Compound (I) and CH-4893237, or a pharmaceutically acceptable salt thereof.
Compound (I) and 11β-fluoro-7α-{5-[methyl(7,7,8,8,9,9,9-heptafluorononyl)amino]pentyl}-17α-methylestra-1,3,5 (10)-triene-3,17-β-diol N-oxide.
Compound (I) and 11β-fluoro-7α-{5-[methyl(8,8,9,9,10,10,10-heptafluorodecyl)amino]pentyl}-17α-methylestra-1,3,5(10)-triene-3,17β-diol N-oxide.
Compound (I) and (RS)-11β-fluoro-7α-{5-[methyl(7,7,8,8,9,9,10,10,10-nonafluorodecyl)amino]pentyl}-17α-methylestra-1,3,5(10)-triene-3,17β-diol N-oxide.
Compound (I) and 11β-fluoro-7α-{5-[methyl(8,8,9,9,9-pentafluorononyl)amino]pentyl}-17α-methylestra-1,3,5(10)-triene-3,17β-diol N-oxide.
Compound (I) and 11β-fluoro-7α-{5-[methyl(9,9,10,10,10-pentafluorodecyl)amino]pentyl-17α-methylestra-1,3,5(10)-triene-3,17β-diol N-oxide.
Compound (I) and 11β-fluoro-7α-{5-[methyl(8,8,9,9,9-pentafluorononyl)amino]pentyl}-17α-methylestra-1,3,5(10)-triene-3,17β-diol.
Compound (I) and 11β-fluoro-7α-{5-[methyl(7,7,8,8,9,9,10,10,10-nonafluorodecyl)amino]pentyl}-17α-methylestra-1,3,5(10)-triene-3,17β-diol.
Compound (I) and 11β-fluoro-7α-{5-[methyl(7,7,8,8,9,9,9-heptafluorononyl)amino]pentyl}-17α-methylestra-1,3,5(10)-triene-3,17β-diol.
Compound (I) and 17α-ethinyl-11β-fluoro-7α-{5-[methyl(7,7,8,8,9,9,10,10,10-nonafluorodecyl)-amino]pentyl}-estra-1,3,5(10)-triene-3,17β-diol.
Compound (I) and 17α-ethinyl-11β-fluoro-3-(2-tetrahydropyranoyloxy)-7α-{5-[methyl(7,7,8,8,9,9,10,10,10-nonafluorodecyl)amino[pentyl}-estra-1,3,5(10)-trien-17β-ol.
Compound (I) and 11β-fluoro-3-(2-tetrahydrophyranyloxy)-7α-{5-methyl(7,7,8,8,9,9,10,10,10-nonafluorodecyl)amino]pentyl 17α-methylestra-1,3,5(10)-trien-17β-ol.
Compound (I) and 11β-fluoro-7α-{5-[methyl(7,7,8,8,9,9,10,10,10-nonafluorodecyl)amino]pentyl}-17α-trifluoromethylestra-1,3,5 (10)-triene-3,17β-diol.
Compound (I) and 11β-fluoro-7α-{5-[methyl(6,6,7,7,8,8,8-heptafluorooctyl)amino[pentyl}-17α-methylestra-1,3,5(5-triene-3,17β-diol.
Compound (I) and 11β-fluoro-7α-{5-[methyl(8,8,9,9,10,10,10-heptafluorodecyl)amino[pentyl}-17α-methylestra-1,3,5(10)-triene-3,17β-diol.
Compound (I) and 11β-fluoro-7α-{5-[methyl(6,6,7,7,8,8,9,9,10,10,10-undecafluorodecyl)amino]-pentyl}-17α-methylestra-1,3,5(10)-triene-3,17β-diol.
Compound (I) and 11β-fluoro-7α-{5-[methyl(5,5,6,6,7,7,8,8,8-nonafluorooctyl)aminopentyl}-17α-methylestra-1,3,5 (10)-triene-3,17β-diol.
Compound (I) and 11β-fluoro-7α-{5-[methyl(9,9,10,10,10-heptafluoroundecyl)amino [pentyl}-17α-methylestra-1,3,5 (10)-triene-3,17β-diol.
Compound (I) and 11β-7α-{5-[methyl(9,9,10,10,10-pentafluorodecyl)amino]pentyl}-17α-methylestra-1,3,5(10)-triene-3,17β-diol.
Compound (I) and 11β-fluoro-7α-{5-[methyl(7,7,8,8,9,9-heptaflorononyl)amino]pentyl}-17α-methylestra-1,3,5(10)-triene-3,17β-diol N-oxide.
Compound (I) and 11β-fluoro-7α-{5-(methyl{3-[(2,3,4,5,6-pentafluorophenyl)sulfanyl]propyl}-amino}pentyl]estra-1,3,5(10)-triene-3,17β-diol N-oxide.
Compound (I) and 11β-fluoro-7α-[5-(methyl{3-[(4,4,5,5,5-pentafluoropentyl)sulfanyl]propyl}amino)-pentyl]estra-1,3,5(10)-triene-3,17β-diol N-oxide.
Compound (I) and 11β-fluoro-7α-[5-(methyl{3-[(4,4,5,5,5-pentafluoropentyl)sulfinyl]propyl}amino)-pentyl]estra-1,3,5(10)-triene-3,17β-diol N-oxide.
Compound (I) and 11β-fluoro-7α-{5-[methyl(7,7,8,8,9,9,10,10,10-nonafluorodecyl)amino]pentyl}estra-1,3,5 (10)-triene-3,17β-diol N-oxide.
Compound (I) and (S)-11β-fluoro-7α-{5-[methyl(7,7,8,8,9,9,10,10,10-nonafluorodecyl)amino]pentyl}-17α-methylestra-1,3,5(10)-triene-3,17β-diol N-oxide.
Compound (I) and (R)-11β-fluoro-7α-{5-[methyl(7,7,8,8,9,9,10,10,10-nonafluorodecyl)amino]pentyl}-17α-methylestra-1,3,5(10)-triene-3,17β-diol N-oxide.
Compound (I) and 11β-fluoro-7α-{5-[methyl(9,9, 10, 10,10-pentylfluorodecyl)amino]pentyl}estra-1,3,5(10)-triene-3,17β-diol N-oxide.
Compound (I) and 11β-fluoro-7α-[5-(methyl{3-[(4,4,5,5,5-pentafluoropentyl)sulfinyl]propyl}amino)-pentyl]estra-1,3,5(10)-trien-3-ol-17-one N-oxide.
Compound (I) and 11β-fluoro-7α-[5-(methyl{3-[(4,4,5,5,5-pentafluoropentyl)sulfanyl]propyl}amino)-pentyl]estra-1,3,5(10)-trien-3-ol-17-one N-oxide.
Compound (I) and 11β-fluoro-7α-{5[methyl(7,7,8,8,9,9,10,10,10-nonafluorodecyl)amino]pentyl}estra-1,3,5 (10)-trien-3-ol-17one N-oxide.

Suitable pharmaceutically-acceptable salts include, for example, salts with alkali metal (such as sodium, potassium or lithium), alkaline earth metals (such as calcium or magnesium), ammonium salts, and salts with organic bases affording physiologically acceptable cations, such as salts with methylamine, dimethylamine, trimethylamine, piperidine and morpholine. In addition, for those compounds which are sufficiently basic, suitable pharmaceutically-acceptable salts include, pharmaceutically-acceptable acid-addition salts with hydrogen halides, sulphuric acid, phosphoric acid and with organic acids such as citric acid, maleic acid, methanesulphonic acid and p-toluenesulphonic acid. Alternatively, the compounds may exist in zwitterionic form.

Suitably Compound (I) is used in the free base form, particularly crystalline Compound (I) Form 1 described in WO2007/010235 and the Cambridge crystallographic database [N-(3-Methoxy-5-methylpyrazin-2-yl)-2-[4-(1,3,4-oxadiazol-2-yl)phenyl]pyridine-3-sulfonamide (ZD4054 Form 1). Acta Crystallographica, Section E: Structure Reports Online (2004), E60(10), o1817-o1819].

Therefore according to the present invention, there is provided a combination, comprising Compound (I) and an aromatase inhibitor for use as a medicament.

Therefore according to the present invention, there is provided a combination, comprising Compound (I) and an estrogen receptor down-regulator for use as a medicament.

Therefore according to the present invention, there is provided a method of treating cancer, in a warm-blooded animal, such as man, in need of such treatment which comprises administering to said animal an effective amount of Compound (I) in combination with an effective amount of an aromatase inhibitor.

Therefore according to the present invention, there is provided a method of treating cancer, in a warm-blooded animal, such as man, in need of such treatment which comprises administering to said animal an effective amount of Compound (I) in combination with an effective amount of an estrogen receptor down-regulator.

For the avoidance of doubt, where the treatment of cancer is indicated, it is to be understood that this also refers to the prevention of metastases and the treatment of metastases, i.e. cancer spread. Therefore the combination of the present invention could be used to treat a patient who has no metastases to stop them occurring, or to lengthen the time period before they occur, and to a patient who already has metastases to treat the metastases themselves. Furthermore the treatment of cancer also refers to treatment of an established primary tumour or tumours and developing primary tumour or tumours. In one aspect of the invention the treatment of cancer relates to the prevention of metastases. In another aspect of the invention the treatment of cancer relates to the treatment of metastases. In another aspect of the invention the treatment of cancer relates to treatment of an established primary tumour or tumours or developing primary tumour or tumours. Herein, the treatment of cancer also refers to the prevention of cancer per se.

In addition the treatment of cancer also refers to the production of an anti-angiogenic effect in a warm blooded animal.

In one embodiment the treatment of cancer relates to an adjuvant treatment. In another embodiment the treatment of cancer refers to the neo-adjuvant treatment of cancer. Accordingly in an embodiment of the invention the combination according to the invention is used as an adjuvant treatment of hormone sensitive breast cancer, particularly as an adjuvant treatment of estrogen receptor positive breast cancer in post-menopausal women. In another embodiment of the invention the combination according to the invention is used as a neo-adjuvant treatment of hormone sensitive breast cancer, particularly as a neo-adjuvant treatment of estrogen receptor positive breast cancer in post-menopausal women. In another embodiment the combination is used to treat advanced (particularly metastatic) hormone sensitive breast cancer, particularly advanced (particularly metastatic) estrogen receptor positive cancer in post-menopausal women.

The term “adjuvant therapy” refers to a treatment given in addition to the primary therapy used to remove or kill the tumour. The adjuvant therapy is used to kill any cancer cells that may have spread from the primary tumour. For breast cancer the primary therapy may be for example, surgery (for example lumpectomy or mastectomy) and/or radiotherapy. The term “neo-adjuvant therapy” refers to a treatment given prior to a primary therapy such as surgery or radiotherapy.

In a further embodiment the treatment of the cancer refers to the first-line treatment of the cancer. In another embodiment the treatment of the cancer refers to the second-line treatment of the cancer (a treatment administered following failure of the first-line treatment of the cancer).

The presence of the estrogen may restore endocrine sensitivity in the tumour and thereby overcome resistance to endocrine therapy in patients with breast cancer.

Therefore, according to a further aspect of the present invention, there is provided a method of treating breast cancer that has become resistant to endocrine therapy, in a warm-blooded animal, such as man, in need of such treatment which comprises administering to said animal an effective amount of a combination comprising an estrogen, Compound (I) and an aromatase inhibitor. Aromatase inhibitors suitable for use in this aspect of the invention are as described hereinbefore.

Therefore, according to a further aspect of the present invention, there is provided a method of treating breast cancer that has become resistant to endocrine therapy, in a warm-blooded animal, such as man, in need of such treatment which comprises administering to said animal an effective amount of a combination comprising an estrogen, Compound (I) and an estrogen receptor down-regulator. Estrogen receptor down-regulator inhibitors suitable for use in this aspect of the invention are as described hereinbefore.

According to another aspect of the present invention the effect of a method of treatment of the present invention is expected to be at least equivalent to the addition of the effects of each of the components of said treatment used alone, that is, of each of Compound (I) and the aromatase inhibitor used alone or Compound (I) and the estrogen receptor down-regulator used alone.

According to another aspect of the present invention the effect of a method of treatment of the present invention is expected to be greater than the addition of the effects of each of the components of said treatment used alone, that is, of each of Compound (I) and the aromatase inhibitor used alone or Compound (I) and the estrogen receptor down-regulator used alone.

According to another aspect of the present invention the effect of a method of treatment of the present invention is expected to be a synergistic effect.

According to the present invention a combination treatment is defined as affording a synergistic effect if the effect is therapeutically superior, as measured by, for example, the extent of the response, the response rate, the time to disease progression or the survival period, to that achievable on dosing one or other of the components of the combination treatment at its conventional dose. For example, the effect of the combination treatment is synergistic if the effect is therapeutically superior to the effect achievable with Compound (I) or the aromatase inhibitor or the estrogen receptor down-regulator alone. Further, the effect of the combination treatment is synergistic if a beneficial effect is obtained in a group of patients that does not respond (or responds poorly) to Compound (I) or the aromatase inhibitor or the estrogen receptor down-regulator alone. In addition, the effect of the combination treatment is defined as affording a synergistic effect if one of the components is dosed at its conventional dose and the other component(s) is/are dosed at a reduced dose and the therapeutic effect, as measured by, for example, the extent of the response, the response rate, the time to disease progression or the survival period, is equivalent to that achievable on dosing conventional amounts of the components of the combination treatment. In particular, synergy is deemed to be present if the conventional dose of Compound (I) or the aromatase inhibitor or the estrogen receptor down-regulator may be reduced without detriment to one or more of the extent of the response, the response rate, the time to disease progression and survival data, in particular without detriment to the duration of the response, but with fewer and/or less troublesome side-effects than those that occur when conventional doses of each component are used.

According to a further aspect of the present invention there is provided a kit comprising Compound (I) and an aromatase inhibitor; optionally with instructions for use.

According to a further aspect of the present invention there is provided a kit comprising Compound (I), and an estrogen receptor down-regulator; optionally with instructions for use.

According to a further aspect of the present invention there is provided a kit comprising:

a) Compound (I), in a first unit dosage form;
b) an aromatase inhibitor; in a second unit dosage form; and
c) container means for containing said first and second dosage forms; and optionally
d) with instructions for use.

An example of a unit dosage form for Compound (I) might be a tablet for oral formulation, see that described herein below. An example of a unit dosage form for an aromatase inhibitor might be a tablet for oral formulation, see that described herein below. An example of a unit dosage from for an estrogen receptor down-regulator might be a formulation for intramuscular administration, see that described herein below.

According to a further aspect of the present invention there is provided a kit comprising:

a) Compound (I), in a first unit dosage form;
b) an estrogen receptor down-regulator; in a second unit dosage form; and
c) container means for containing said first and second dosage forms; and optionally
d) with instructions for use.

According to a further aspect of the present invention there is provided a kit comprising:

a) Compound (I), together with a pharmaceutically acceptable diluent or carrier, in a first unit dosage form;
b) an aromatase inhibitor, in a second unit dosage form; and
c) container means for containing said first and second dosage forms; and optionally
d) with instructions for use.

According to a further aspect of the present invention there is provided a kit comprising:

a) Compound (I), together with a pharmaceutically acceptable diluent or carrier, in a first unit dosage form;
b) an estrogen receptor down-regulator, in a second unit dosage form; and
c) container means for containing said first and second dosage forms; and optionally
d) with instructions for use.

The combination may be used as a single composition containing Compound (I) and the aromatase inhibitor or the estrogen receptor down regulator.

Accordingly, a further aspect of the invention provides a pharmaceutical composition which comprises Compound (I) and an aromatase inhibitor in association with a pharmaceutically acceptable diluent or carrier.

According to a further aspect of the invention there is provided a pharmaceutical composition which comprises Compound (I) and an estrogen receptor down-regulator in association with a pharmaceutically acceptable diluent or carrier.

According to a further aspect of the invention there is provided a pharmaceutical composition which comprises Compound (I) and an aromatase inhibitor in association with a pharmaceutically acceptable diluent or carrier for use in the treatment of cancer.

According to a further aspect of the invention there is provided a pharmaceutical composition which comprises Compound (I) and an estrogen receptor down-regulator in association with a pharmaceutically acceptable diluent or carrier for use in the treatment of cancer.

The combination may be used as two individual compositions, one containing Compound (I) and one containing the aromatase inhibitor or estrogen receptor down-regulator, wherein the two compositions are administered simultaneously, separately or sequentially.

Accordingly, a further aspect of the invention provides a pharmaceutical composition which comprises Compound (I), in association with a pharmaceutically acceptable diluent or carrier, in combination with a pharmaceutical composition which comprises an aromatase inhibitor in association with a pharmaceutically acceptable diluent or carrier.

According to a further aspect of the invention there is provided a pharmaceutical composition which comprises Compound (I), in association with a pharmaceutically acceptable diluent or carrier, in combination with a pharmaceutical composition which comprises an estrogen receptor down-regulator in association with a pharmaceutically acceptable diluent or carrier.

According to a further aspect of the invention there is provided a pharmaceutical composition which comprises Compound (I), in association with a pharmaceutically acceptable diluent or carrier, in combination with a pharmaceutical composition which comprises an aromatase inhibitor in association with a pharmaceutically acceptable diluent or carrier for use in the treatment of cancer.

According to a further aspect of the invention there is provided a pharmaceutical composition which comprises Compound (I), in association with a pharmaceutically acceptable diluent or carrier, in combination with a pharmaceutical composition which comprises an estrogen receptor down-regulator in association with a pharmaceutically acceptable diluent or carrier for use in the treatment of cancer.

The pharmaceutical compositions may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository. In general the above compositions may be prepared in a conventional manner using conventional excipients.

For example Compound (I) can be formulated as a tablet using the following excipients:
Compound (I);
Lactose monohydrate (filler);
Croscarmellose sodium (disintegrant);
Povidone (binder);
Magnesium stearate (lubricant);
Hypromellose (film coat component);
Polyethylene glycol 300 (film coat component); and
Titanium dioxide (film coat component).

Alternatively, Compound (I) can be formulated as a tablet comprising Compound (I), mannitol and microcrystalline cellulose. For example, a tablet containing 10 mg of Compound (I) shown in Table 1:

TABLE 1
Active tablet (10 mg, mannitol/microcrystalline cellulose filler)

| Ingredient | mg/tablet | | Function |
|---|---|---|---|
| Tablet core | | % of core weight | |
| Compound (I) | 10.000 | 6.67 | Drug substance |
| Mannitol | 110.750 | 73.83 | Filler |
| Microcrystalline | 18.750 | 12.50 | Filler |
| cellulose | | | |
| Croscarmellose Na | 4.500 | 3.00 | Disintegrant |
| Povidone K29/32 | 4.500 | 3.00 | Binder |
| Magnesium stearate | 1.500 | 1.00 | Lubricant |
| Core tablet weight | 150.000 | | |
| Tablet coating | | % of coating weight | |
| Hypromellose 2910 | 3.281 | 62.50 | Film former |
| Titanium dioxide | 1.563 | 29.77 | Opacifier |
| Polyethylene | 0.328 | 6.25 | Plasticiser |
| glycol 400 | | | |
| Iron oxide yellow | 0.059 | 1.12 | Colouring agent |
| Iron oxide red | 0.014 | 0.27 | Colouring agent |
| Iron oxide black | 0.004 | 0.08 | Colouring agent |
| | | % of core weight | |
| Nominal coating | 5.250 | 3.50 | |
| weight | | | |

The tablets may be prepared using, for example, a wet granulation process as described below.

Compound (I) (1.334 kg), mannitol (Partek M200, Merck, 14.76 kg), microcrystalline cellulose (Avicel PH101, FMC, 2.5 kg), croscarmellose sodium (Ac-Di-Sol, FMC, 600 g) and poyvinylpyrrolidinone (Plasdone K29/32, ISP, 600 g) are mixed together in a Vector GMX75 high shear blender. Water (4.5 kg, addition rate of 1.2 kg/minute) is sprayed into the mixture and the mixture granulated for about 5 minutes. The granules are dried in an O'Hara 30/60 fluid bed dryer (inlet air temperature 70° C., air flow rate sufficient to fluidise the granule bed) to a moisture content of <2% w/w and the dried granules milled using a Quadro Co mil 194 (screen mesh 0.062 inches (1.6 mm), 400 rpm).

Four of the above portions are combined and 800 g magnesium stearate added. The 80 kg batch is transferred to a Pharmatech BV400 blender and the mixture blended. The mixture is then compressed into tablets (150 mg compression weight, plain, round, bi-convex 7 mm diameter) using an IMA Kilian Synthesis 500 tablet press (80,000 tablets per hour, 7.5 kN compression force). The tablets are then coated using a Manesty Premier 200 coater with Opadry Beige (Colorcon 03B27164, 315 g/kg aqueous solution). The total coating solution applied is equivalent to 35 g/kg of Opadry per mass of tablet cores.

In those embodiments where Compound (I) is used in combination with the estrogen receptor down-regulator fulvestrant, the fulvestrant may be formulated as a composition suitable for intramuscular administration, for example a composition comprising fulvestrant in a ricinoleate vehicle, a pharmaceutically acceptable non-aqueous ester solvent, and a pharmaceutically acceptable alcohol. Particular fulvestrant compositions are those described in U.S. Pat. No. 6,774,122.

According to a further aspect of the present invention there is provided a kit comprising Compound (I) and an aromatase inhibitor; optionally with instructions for use; for use in the treatment of cancer.

According to a further aspect of the present invention there is provided a kit comprising Compound (I), and an estrogen receptor down-regulator; optionally with instructions for use; for use in the treatment of cancer.

According to a further aspect of the present invention there is provided a kit comprising:

a) Compound (I), in a first unit dosage form;
b) an aromatase inhibitor in a second unit dosage form; and
c) container means for containing said first and second dosage forms; and optionally
d) with instructions for use; for use in the treatment of cancer.

According to a further aspect of the present invention there is provided a kit comprising:

a) Compound (I), in a first unit dosage form;
b) an estrogen receptor down-regulator in a second unit dosage form; and
c) container means for containing said first and second dosage forms; and optionally
d) with instructions for use;
for use in the treatment of cancer.

According to a further aspect of the present invention there is provided a kit comprising:

a) Compound (I), together with a pharmaceutically acceptable diluent or carrier, in a first unit dosage form;
b) an aromatase inhibitor in a second unit dosage form; and
c) container means for containing said first and second dosage forms; and optionally
d) with instructions for use; for use in the treatment of cancer.

According to a further aspect of the present invention there is provided a kit comprising:

a) Compound (I), together with a pharmaceutically acceptable diluent or carrier, in a first unit dosage form;
b) an estrogen receptor down-regulator in a second unit dosage form; and
c) container means for containing said first and second dosage forms; and optionally
d) with instructions for use; for use in the treatment of cancer.

According to a further aspect of the present invention there is provided a combination comprising Compound (I) and an aromatase inhibitor for use in the treatment of cancer.

According to a further aspect of the present invention there is provided a combination comprising Compound (I), and an estrogen receptor down-regulator for use in the treatment of cancer.

According to a further aspect of the present invention there is provided a combination treatment comprising the administration of an effective amount of Compound (I), optionally together with a pharmaceutically acceptable diluent or carrier, in combination with an effective amount of an aromatase inhibitor optionally together with a pharmaceutically acceptable diluent or carrier to a warm-blooded animal, such as man in need of such therapeutic treatment for use in the treatment of cancer.

According to a further aspect of the present invention there is provided a combination treatment comprising the administration of an effective amount of Compound (I), optionally together with a pharmaceutically acceptable diluent or carrier, in combination with an effective amount of an estrogen receptor down-regulator optionally together with a pharmaceutically acceptable diluent or carrier to a warm-blooded animal, such as man in need of such therapeutic treatment for use in the treatment of cancer.

The amount of Compound (I), or a pharmaceutically acceptable salt thereof, administered would be that sufficient to provide the desired pharmaceutical effect. For instance, Compound (I) could be administered to a warm-blooded animal orally, at a unit dose less than 1 g daily but more than 2.5 mg. Particularly Compound (I) could be administered to a warm-blooded animal, at a unit dose of less than 250 mg per day. In another aspect of the invention, Compound (I) could be administered to a warm-blooded animal, at a unit dose of less than 130 mg per day. In a further aspect of the invention, Compound (I) could be administered to a warm-blooded animal, at a unit dose of less than 50 mg per day. Particularly Compound (I) could be administered to a warm-blooded animal, at a unit dose of 10 mg per day. In another aspect of the invention, particularly Compound (I) could be administered to a warm-blooded animal, at a unit dose of 15 mg per day.

Aromatase inhibitors would normally be administered to a warm-blooded animal at a unit dose, of an amount known to the skilled practitioner as a therapeutically effective dose. For a single dosage form, the active ingredients may be compounded with an appropriate and convenient amount of excipients which may vary from about 5 to about 98 percent by weight of the total composition. Dosage unit forms will generally contain about 1 mg to about 20 mg, particularly 1 mg to 5 mg of each active ingredient. However the daily dose will necessarily be varied depending upon the host treated, the particular route of administration, and the severity of the illness being treated. Accordingly the optimum dosage may be determined by the practitioner who is treating any particular patient.

The estrogen receptor down-regulator will normally be administered to a warm-blooded animal at a unit dose, of an amount known to the skilled practitioner as a therapeutically effective dose. For a single dosage form, the active ingredients may be compounded with an appropriate and convenient amount of excipients which may vary from about 5 to about 98 percent by weight of the total composition. Dosage unit forms will generally contain about 20-500 mg, particularly 250 mg, of each active ingredient. However the daily dose will necessarily be varied depending upon the host treated, the particular route of administration, and the severity of the illness being treated. Accordingly the optimum dosage may be determined by the practitioner who is treating any particular patient.

The dosage of each of the drugs and their proportions have to be composed so that the best possible treatment effects, as defined by national and international guidelines (which are periodically reviewed and re-defined), will be met.

### EXAMPLES

The invention is further illustrated by way of the following examples, which are intended to elaborate several embodiments of the invention. These examples are not intended to, nor are they to be construed to, limit the scope of the invention. It will be clear that the invention may be practiced otherwise than as particularly described herein. Numerous modifications and variations of the present invention are possible in view of the teachings herein and, therefore, are within the scope of the invention.

Compound (I) was used as Compound (I) Form 1 described in WO2007/010235. Fulvestrant, anastrozole and letrozole are commercially available.

### EXPERIMENTAL

The in-vitro studies described below were carried out to demonstrate the effect of ET-1, aromatase and estrogens and combinations according to the invention on migration and invasion of breast cancer cells.

The in-vivo xenograft study described below was carried out to demonstrate the effect of combinations according to the invention on inhibiting tumour growth of a breast cancer cell line.

### Methodology

### In-Vitro Studies

The well-established human breast cancer cell lines MCF-7 [1], and the aromatase over-expressing derivatives of this cell line, MCF-7aro [2], [3] were used for all in vitro experiments. MCF-7 and MCF-7aro cells were maintained in RPMI 1640 containing 10% foetal calf serum, 1% glutamine and 1% penicillin/streptomycin; for MCF-7aro media contained 600 μg/ml G418 (geneticin disulfate; Sigma-Aldrich). Cell culture media and FCS were obtained from Gibco (Karlsruhe, Germany). MCF-7 and MCF-7aro cells were kept in a humidified atmosphere at 37° C. of 5% CO₂.

MCF-7 and MCF-7aro cell migration assays were performed in triplicate using Falcon® Cell Culture Inserts (BD Biosciences, Bedford, Mass., USA). Cells (2.5×10⁴ cells/well) were added to the upper compartment in serum-free RPMI medium. RPMI supplemented with 10% foetal calf serum and 25 ng/ml bFGF was given to the lower compartment. For incubation with single agents, either endothelin receptor antagonist (Compound (I)), aromatase inhibitor (anastrozole or letrozole) or estrogen receptor down-regulator (fulvestrant), was added to both insert and lower compartment at 100 nM (Compound (I)), 1 μM (anastrozole) 100 nM (fulvestrant) and 100 nM (letrozole). For incubation with combinations of, either endothelin antagonist with aromatase inhibitor or endothelin antagonist with estrogen receptor down-regulator, these were added simultaneously to both insert and lower compartment at concentrations indicated above. Assays with MCF-7aro cells were supplemented with 100 nM A4-Androsten-3,17-dione. Assays with estrogen receptor down-regulator were supplemented with 17β-estradiol (10 nM). Cells then were cultured under routine conditions and allowed to migrate for 24 hours. Subsequently, the medium was removed from the insert and cells on the upper side of the membrane were removed by scraping. Cells on the lower surface were fixed with methanol, stained using standard techniques and counted under microscopy. Relative migration was expressed as a percentage of the cell number on compound-treated inserts compared to controls.

For invasion assays, BD BioCoat® Matrigel Invasion Chambers (BD Biosciences, Bedford, Mass., USA) were used, in which a layer of matrigel matrix serves as a reconstituted basement membrane in vitro. 0.5 ml of MCF-7 cell suspension in serum-free RPMI (5×10⁴ cells/ml) was added to the insert, while RPMI supplemented with 10% foetal calf serum and 25 ng/ml bFGF was given to the lower compartment. For assessment of single agents in test cells, either the endothelin antagonist, or estrogen receptor antagonist was added at 100 nM (Compound (I), fulvestrant) to either compartment together with 17β-estradiol (10 nM); for assessment of combinations of endothelin antagonist with estrogen receptor down-regulator, these were added simultaneously at concentrations indicated above following pre-addition of 17β-estradiol; for control cells, either pure solvent or 17-β-estradiol was added instead. Cells were then incubated for 48 hours at cell culture conditions. After incubation, non-invading cells were removed from the upper surface by scraping. The cells on the lower surface were fixed, stained and counted by photographing the membrane through the microscope at 40× magnification. At each triplicate membrane, cells in five different fields were counted. Relative invasiveness was expressed as percentage of the cell number on compound-treated inserts compared to control inserts.

In the in-vitro studies Compound (I) was dissolved in DMSO (final DMSO concentration in the assay less than 0.5%). Fulvestrant, letrozole and anastozole were dissolved in a 98% ethanol (final ethanol concentration in the assay less than 0.1%). The same respective concentrations of DMSO and/or ethanol were used in the control assays.

### In-Vitro Results

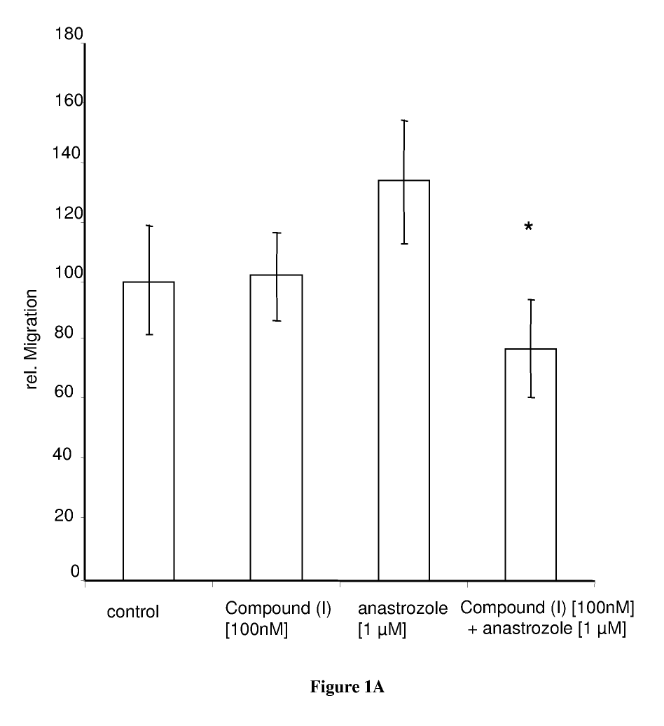

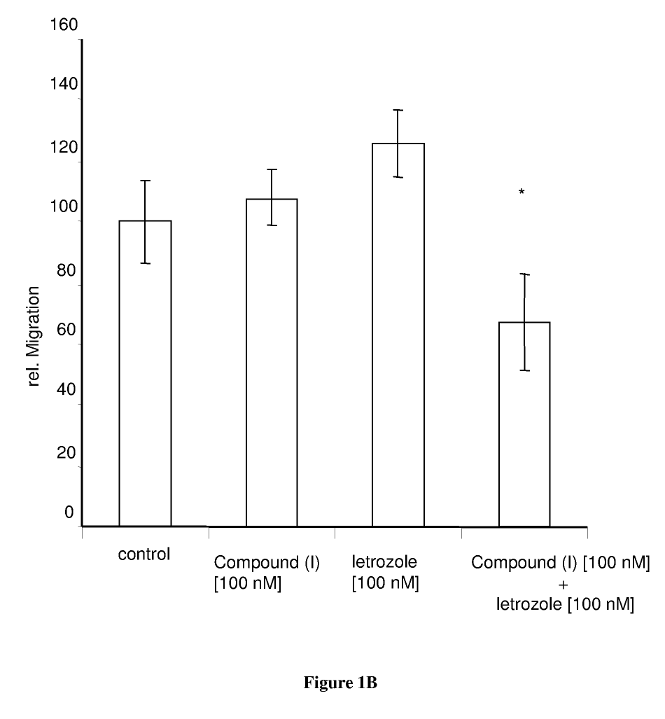

The results of the in-vitro studies are summarised in FIGS. 1 to 3.

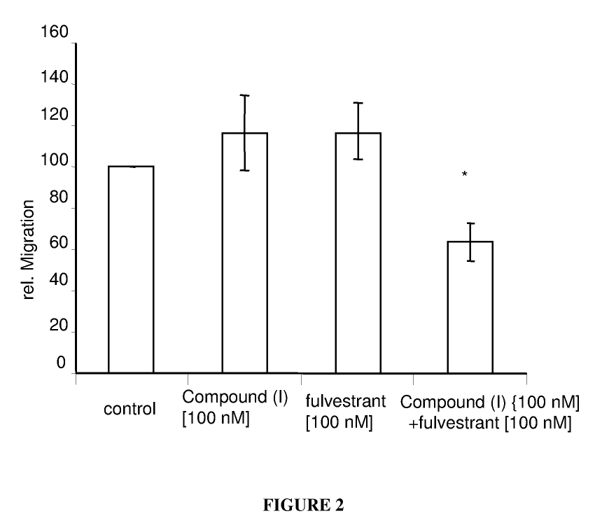

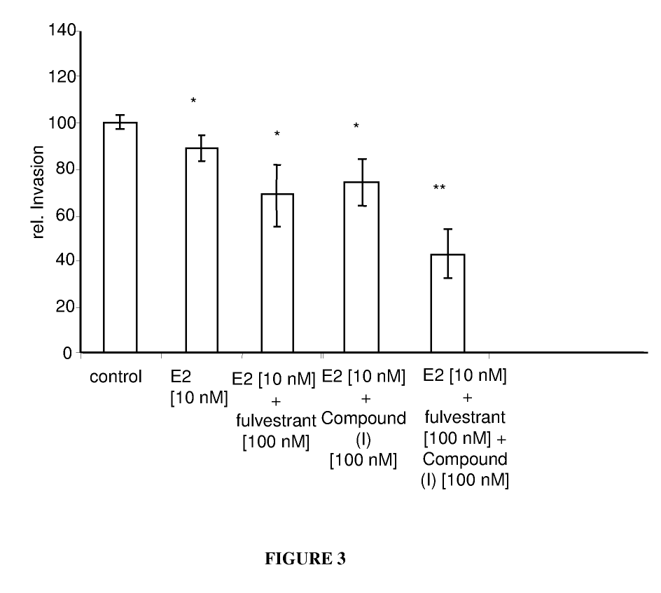

The results show that the combinations according to the invention significantly reduced migration and invasion of the breast cancer cells in-vitro. FIGS. 1 and 2 illustrate that the combinations according to the invention exhibit a statistically significant, synergistic, effect on cell migration. FIG. 3 illustrates that a combination of compound (I) and fulvestrant exhibits an enhanced, at least an additive, effect upon cell invasion of the MCF-7 cells.

### In-Vivo Xenograft Study

For in-vivo combination assays, xenografts of MCF-7-Ac1 [4] breast cancer cells were used. Prior to tumour cell injection and treatment, animals were ovariectomized. Subsequently, cells were injected subcutaneously (3×10⁶ cells in 100 μl matrigel). All animals were supplemented with Δ4-Androsten-3,17-dione by subcutaneous injection. Beginning on the day of tumour cell injection, animals received oral application of either Compound (I) in aqueous suspension (containing 1% polysorbate tween 80; 30 mg/kg, once daily), anastrozole (aqueous suspension containing 1% polysorbate tween 80; 0.5 kg, once daily), fulvestrant (250 mg/kg, Faslodex™ once weekly) or vehicle only (controls, once daily) or Compound (I) (30 mg/kg aqueous suspension containing 1% polysorbate tween 80, once daily) in combination with either anastrozole (aqueous suspension containing 1% polysorbate tween 80; 0.5 mg/kg, once daily) or fulvestrant (250 mg/kg, Faslodex™ once weekly)

### In-Vivo Results

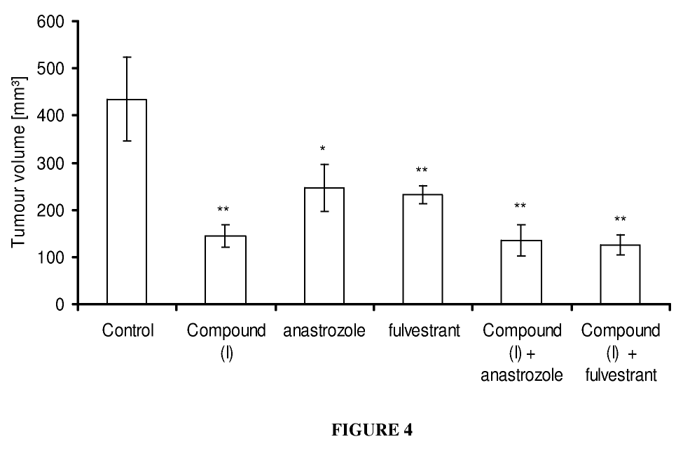

FIG. 4 shows the results of the xenograft study 45 days after injection with the MCF-7-Acl cells. FIG. 4 illustrates that the anti-tumour effect of anastrozole and fulvestrant are enhanced when used in combination with Compound (I) in this xenograft model.

### CONCLUSIONS

The results show that in-vitro, in aromatase over-expressing breast cancer cells (MCF-7aro), combinations of an endothelin receptor antagonist (Compound (I)) with an aromatase inhibitor or estrogen receptor down-regulator significantly reduces migration of these cells. Moreover, in non-aromatase-sensitive breast cancer cells (MCF-7), combinations of an endothelin receptor antagonist with an estrogen receptor down-regulator, significantly reduced both cellular migration and invasion.

Furthermore, we have shown that in-vivo in a post-menopausal nude mouse xenograft model carrying tumour grown from aromatase over-expressing MCF-7aro breast cancer cells, combinations of the endothelin receptor antagonist (Compound (I)) with an aromatase inhibitor or an estrogen receptor down-regulator enhances the anti-tumour effect of the aromatase inhibitor or estrogen receptor down-regulator.

Therefore, an endothelin receptor antagonist and either aromatase inhibitor or estrogen receptor down-regulator combination therapy should have a beneficial effect in preventing pathological breast cancer cell changes and may be useful in the treatment of cancers such as breast cancer.

### REFERENCES

[1] Brooks S C, Locke E R, Soule H D (1973) Estrogen receptor in a human cell line (MCF-7) from breast carcinoma. J Biol Chem 248: 6251-6253.
[2] Sun X Z, Zhou D, Chem S (1997) Autocrine and paracrine actions of breast tumor aromatase. A three-dimensional cell culture study involving aromatase transfected MCF-7 and T-47D cells. J Steroid Biochem Mol Biol 63: 29-36.
[3] Zhou D J, Pompon D, Chen S A (1990) Stable expression of human aromatase complementary DNA in mammalian cells: a useful system for aromatase inhibitor screening. Cancer Res 50: 6949-6954.
[4] Brodie A, Jelovac D, Long B J (2003) Predictions from a preclinical model: studies of aromatase inhibitors and antiestrogens. Clin Cancer Res 9: 455-459.

1. A combination, comprising N-(3-methoxy-5-methylpyrazin-2-yl)-2-(4-[1,3,4-oxadiazol-2-yl]phenyl)pyridine-3-sulphonamide, or a pharmaceutically acceptable salt thereof, and an aromatase inhibitor.

2. A combination, comprising N-(3-methoxy-5-methylpyrazin-2-yl)-2-(4-[1,3,4-oxadiazol-2-yl]phenyl)pyridine-3-sulphonamide, or a pharmaceutically acceptable salt thereof, and an estrogen receptor down-regulator.

3. A combination as claimed in claim 1 wherein the aromatase inhibitor is anastrozole.

4. A combination as claimed in claim 1 wherein the aromatase inhibitor is exemestane.

5. A combination as claimed in claim 1 wherein the aromatase inhibitor is letrozole.

6. A combination as claimed in claim 2 wherein the estrogen receptor down-regulator is fulvestrant.

7. A combination as claimed in claim 1 or claim 2 for use as a medicament.

8. A pharmaceutical composition comprising a combination as claimed in claim 1 or claim 2 in association with a pharmaceutically acceptable diluent or carrier.

9. A method of treating cancer, in a warm-blooded animal, such as man, in need of such treatment which comprises administering to said animal an effective amount of a combination as claimed in claim 1 or claim 2.

10. A method as claimed in claim 9 wherein the cancer is breast cancer.

11. A method as claimed in claim 9 wherein the cancer is in a metastatic state.

12. A method as claimed in claim 9 wherein the cancer is metastatic breast cancer.
